(19) United States
(12) Patent Application Publication
(10) Pub. No.: US 2021/0180801 A1
(43) Pub. Date: Jun. 17, 2021

(54) AIR PURIFICATION-AROMATHERAPY DEVICE

(71) Applicant: PUZHEN LIFE CO., LTD., Shatin (HK)

(72) Inventor: Andy LEE, Shatin (HK)

(21) Appl. No.: 16/833,264

(22) Filed: Mar. 27, 2020

(30) Foreign Application Priority Data
Dec. 17, 2019 (CN) 201922278991.0

(51) Int. Cl. F24F 1/008; B01D 46/00

(52) U.S. Cl. CPC F24F 1/008; A61L 9/14; B01D 46/0038

(57) ABSTRACT

An air purification-aromatherapy device comprises: a housing, a fan, a filter, a vial and an atomizer. The housing is provided with an air inlet and an air outlet configured for air circulation, and the housing is provided with an outlet orifice for spraying the atomized essential oil out of the housing. The fan is disposed within the housing, the filter is arranged at the air inlet, the vial is detachably arranged in the housing, and the atomizer is disposed within the housing. When in use, the essential oil in the vial is atomized and then sprayed through the atomizer; a negative pressure airflow is generated, such that the air in the vehicle is sucked into the housing, and filtered and purified by the filter, and then the filtered and purified air is discharged from the air outlet of the housing to the vehicle.

### CROSS-REFERENCE TO RELATED APPLICATION

The present application claims priority to Chinese Patent Application Ser. No. CN201922278991.0, filed on Dec. 17, 2019, the entire content of which is incorporated herein by reference in its entirety.

### TECHNICAL FIELD

The present application relates to the field of air purification technology, and more particularly relates to an air purification-aromatherapy device.

### BACKGROUND

With the continuous improvement of people's living standards, more and more motorists want to avoid the deterioration of air quality in enclosed vehicle spaces due to factors such as human perspiration, gas emissions from materials in the vehicle, or bacterial growth. At present, people generally place perfumes or aromatherapy machines in vehicles, so as to release aromas or diffuse atomized essential oil, thereby achieving the purpose of improving the air quality in vehicles. However, the aromas released by the perfumes or the atomized essential oil diffused by the aromatherapy machine often reacts with human sweat and impurity gases in the environment, thereby generating awful odors, which affects the user experience.

### SUMMARY

The objective of the present application is to provide an air purification-aromatherapy device, so as to solve the problem in the related art that the perfume or the aromatherapy machine has a poor effect on improving the air quality in the vehicle.

To achieve said objective, the technical solution adopted in the embodiments of the present application is to provide an air purification-aromatherapy device, including:

a housing, provided with an air inlet and an air outlet;

a fan, disposed within the housing;

a filter, arranged at the air inlet;

a vial, detachably arranged in the housing for storing essential oil; and

an atomizer, configured for atomizing the essential oil in the vial and spraying out an atomized essential oil;

wherein the atomizer is disposed within the housing, and the housing is provided with an outlet orifice for spraying the atomized essential oil out of the housing.

In an embodiment, the filter is cylindrical, an outer peripheral surface of the filter is affixed to an inner sidewall of the housing, and the air inlet is defined in a sidewall of the housing.

In an embodiment, the air inlet comprises a plurality of air inlet holes.

In an embodiment, an outer contour of the housing is tubular, the housing comprises an atomizing tube segment, a filtering tube segment detachably connected to the atomizing tube segment at one end, a top cover covering one end of the atomizing tube segment that is far from the filtering tube segment, and a bottom cover covering one end of the filtering tube segment that is far from the atomizing tube segment, the atomizer and the vial are placed in the atomizing tube segment, the fan and the filter are placed in the filtering tube segment, the air outlet and the outlet orifice are arranged on the top cover, and the air inlet is arranged on the filtering tube segment.

In an embodiment, an outer diameter of the atomizing tube segment is larger than an outer diameter of the filtering tube segment.

In an embodiment, the housing further comprises a positioning tube segment configured for cooperatively connecting the filtering tube segment, one end of the positioning tube segment is connected to the atomizing tube segment, and one end of the filtering tube segment is sleeved on the positioning tube segment.

In an embodiment, the atomizer comprises an air pump for providing an airflow, an atomizing hood provided with an atomizing chamber, a gas nozzle placed in the atomizing chamber, an air tube connecting the air pump and the gas nozzle, an oil suction tube extending into the vial, and an oil nozzle connected to the oil suction tube , the atomizing hood is provided with a connecting port for cooperatively connecting the vial, the connecting port is in communication with the atomizing chamber, an air outlet of the gas nozzle is located at a corresponding position of an oil outlet of the oil nozzle, and the atomizing chamber is in communication with the outlet orifice.

In an embodiment, an outer sidewall of a vial mouth of the vial is provided with external threads, and an inner sidewall of the connecting port is provided with internal threads that match with the external threads.

In an embodiment, a bottom of the atomizing chamber has an inclined surface extending obliquely to the connecting port.

In an embodiment, the housing is provided with an accommodation chamber therein for accommodating and positioning the vial.

The one or more technical solutions in the embodiments of the present application have at least one of the following technical effects:

The beneficial effects of the air purification-aromatherapy device provided in the present application is that: compared with the prior art, the air purification-aromatherapy device according to the present application is provided with a vial and an atomizer in the housing, such that the essential oil stored in the vial is atomized and then sprayed through the atomizer, and the atomized essential oil particles are diffused into the air inside the vehicle through the outlet orifice on the housing, which plays a role of incense to the air inside the vehicle. In addition, a fan and a filter are arranged in the housing, a negative pressure airflow is generated in the housing by the fan, such that the air in the vehicle is sucked into the housing from the air inlet of the housing, and filtered and purified by the filter, and then the filtered and purified air is discharged from the air outlet of the housing to the vehicle, thereby realizing the circulation and purification of the air in the vehicle. In this way, the air in the vehicle can be scented and purified, and the air quality in the vehicle can be better improved.

### BRIEF DESCRIPTION OF THE DRAWINGS

In order to more clearly explain the technical solutions in the embodiments of the present application, the drawings used in the embodiments or the description of the prior art will be briefly introduced below. It is obvious that the drawings in the following description are only some embodiments of the present application. For those of ordinary skill in the art, other drawings may be obtained based on these drawings without paying creative labor.

FIG. 1 is a perspective view showing an air purification-aromatherapy device according to an embodiment of the present application;

FIG. 2 is a cross-sectional view the air purification-aromatherapy device;

FIG. 3 is a partially enlarged view of an atomizer of the air purification-aromatherapy device;

FIG. 4 is an exploded view of the air purification-aromatherapy device; and

FIG. 5 is an enlarged view showing an gas nozzle and an oil nozzle of the air purification-aromatherapy device.

Reference numerals in the drawings are as follows:

1-housing; 11-air inlet; 12-air outlet; 13- outlet orifice; 14- atomizing tube segment; 15- filtering tube segment; 16-top cover; 17-bottom cover; 18-positioning tube segment;

2-fan;

3-filter;

4-vial;

5-atomizer; 51-air pump; 52-atomizing hood; 521-atomizing chamber; 522-connecting port; 53-gas nozzle; 54-oil nozzle; 55-oil suction tube;

6- accommodation chamber.

### DETAILED DESCRIPTION

In order to make the technical problems, technical solutions and beneficial effects of the present application to be more clearly understood, the present application will be further described in detail with reference to the accompanying drawings and embodiments. It should be understood that the specific embodiments described herein are only used to explain the present application and are not intended to limit the present application.

It should be noted that when an element is referred to as being “connected to” or “disposed/placed/arranged on/in” another element, the element may be directly or indirectly on the other element. When an element is referred to as being “connected to” another element, the element may be directly or indirectly connected to the other element.

In addition, the terms “first” and “second” are used for descriptive purposes only and cannot be understood as indicating or implying relative importance or implicitly indicating the number of technical features indicated. Therefore, the features defined as “first” and “second” may explicitly or implicitly include one or more of the features. In the description of the present application, the meaning of “a plurality of” is two or more, unless it is specifically defined otherwise. The meaning of “several” is one or more, unless it is specifically defined otherwise.

In the description of the present application, it should be noted that the terms “install”, “mount”, “connect”, and “fix” should be understood in a general sense, unless they are explicitly stated and limited otherwise. For example, they may be fixed connections, removable connections, or integral connections; they may be mechanical connections, or electrical connections; they may be directly connected, or indirectly connected through an intermediate medium; they may be internal connections of two elements, or interactions between two elements. For those of ordinary skill in the art, the specific meanings of the above-said terms in the present application may be understood according to specific situations.

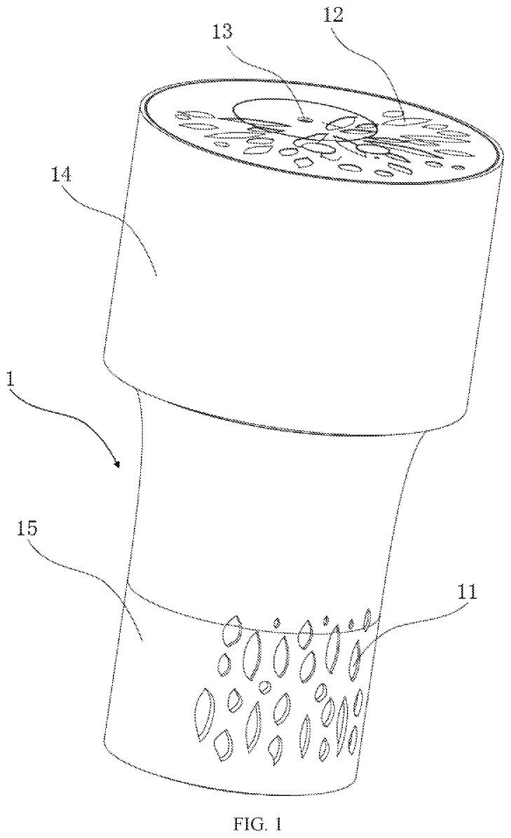

Referring to FIGS. 1-3, the air purification-aromatherapy device provided in the present application will be described. The air purification-aromatherapy device provided in the present application includes a housing 1, a fan 2, a filter 3, a vial 4 and an atomizer 5. The housing 1 defines an air inlet 11, and an air outlet 12 configured for air circulation. The housing 1 is provided with an outlet orifice 13 for spraying an atomized essential oil out of the housing 1. The fan 2 is disposed within the housing 1 and the filter 3 is disposed at the air inlet 11. A negative pressure airflow is generated in the housing 1 by the fan 2. Under the negative pressure airflow, the external air will be sucked in from the air inlet 11 of the housing 1, and then discharged from the air outlet 12 of the housing 1, so as to realize air circulation. Moreover, the filter 3 filters the air sucked into the housing 1 to purify the air in the vehicle. The vial 4 is detachably arranged in the housing 1, and the atomizer 5 is disposed within the housing 1. The essential oil in the vial 4 is atomized and then sprayed through the atomizer 5, and the atomized essential oil particles are diffused into the air inside the vehicle through the outlet orifice 13, which plays a role of incense to the air in the vehicle.

Compared with the prior art, the air purification-aromatherapy device provided in the present application is provided with a vial 4 and an atomizer 5 in the housing 1. The essential oil in the vial 4 can be atomized and then sprayed out by the atomizer 5, and the atomized essential oil particles are diffused into the air in the vehicle through the outlet orifice 13 on the housing 1, which plays a role of incense to the air in the vehicle. In addition, a fan 2 and a filter 3 are disposed within the housing 1. A negative pressure airflow is generated in the housing 1 through the fan 2. The air in the vehicle is sucked into the housing 1 through the air inlet 11 of the housing 1, and filtered and purified by the filter 3, and then the filtered and purified air is discharged into the vehicle from the air outlet 12 of the housing 1, thereby realizing the circulation and purification of the air in the vehicle. In this way, the air in the vehicle can be scented and purified, and the air quality in the vehicle can be improved better.

Specifically, the filter 3 may include, but is not limited to, an electrostatic electret strainer with low wind resistance, high efficiency, and high dust holding capacity, an activated carbon strainer with large surface area due to its developed void structure, or an high efficiency particulate air filter (HEPA strainer).

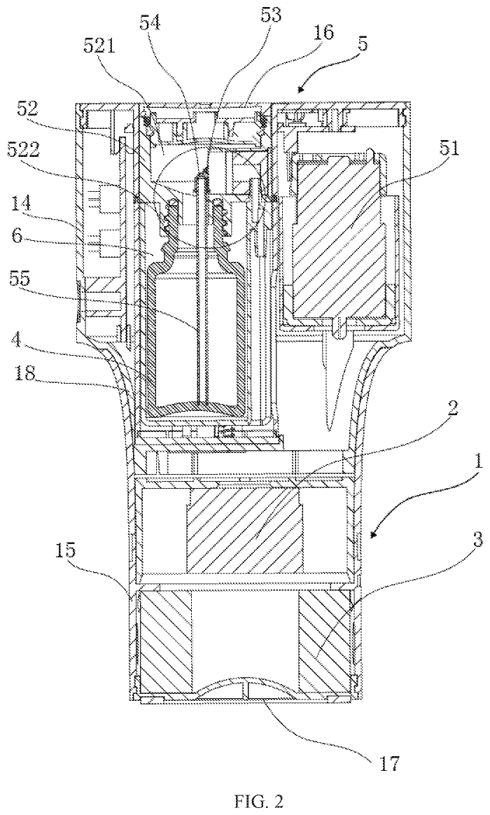

According to an embodiment, referring to FIGS. 2-4, as a specific implementation of the air purification-aromatherapy device provided in the present application, the filter 3 is cylindrical, and an outer peripheral surface of the filter 3 is attached to an inner sidewall of the housing 1, and the air inlet 11 is arranged on a sidewall of the housing 1.

In this embodiment, the air inlet 11 is defined in the sidewall of the housing 1, so as to facilitate the housing 1 to suck in the air in the vehicle, thereby increasing the air flow into the housing 1. In addition, a cylindrical filter 3 is disposed at a corresponding position of the air inlet 11, so as to increase a contact area between the airflow and the filter 3, thereby effectively improving the air filtration efficiency. In addition, the airflow converges from an outer peripheral wall of the cylindrical filter 3 into the cylindrical filter 3, which is conducive to reducing the airflow resistance, facilitates the airflow to pass through the filter 3 quickly, and further improves the air filtration efficiency.

According to an embodiment, referring to FIGS. 2-4, as a specific implementation of the air purification-aromatherapy device provided in the present application, the air inlet 11 includes a plurality of air inlet holes.

In this embodiment, the sidewall of the housing 1 defines a plurality of air inlet holes, and the plurality of air inlet holes form the air inlet 11. It is beneficial for the airflow to enter the housing 1 dispersedly, thereby avoiding excessively concentrated airflow to the cylindrical filter 3, which impacts the cylindrical filter 3 and affects the filtrating effect.

Preferably, as a specific implementation of the air purification-aromatherapy device provided in the present application, a plurality of air inlet holes are evenly distributed on the sidewall of the housing 1, which is beneficial for the airflow to enter the housing 1 uniformly and facilitates the cylindrical filter 3 efficiently filters the uniform and dispersed airflow, thereby improving the air purification effect to a certain extent.

According to an embodiment, referring to FIGS. 1-3, as a specific implementation manner of the air purification-aromatherapy device provided in the present application, an outer contour of the housing 1 is tubular. The housing 1 includes an atomizing pipe segment. 14, a filtering tube segment 15 detachably connected to the atomizing tube segment 14 at one end, a top cover 16 covering one end of the atomizing tube segment 14 that is away from the filtering tube segment 15, and a bottom cover 17 covering one end of the filtering tube segment 15 that is away from the atomizing tube segment 14. The atomizer 5 and the vial 4 are placed in the atomizing tube segment 14. The fan 2 and the filter 3 are placed in the filtering tube segment 15. The air outlet 12 and the outlet orifice 13 are arranged on the top cover 16, and the air inlet 11 is arranged on the filtering tube segment 15.

In this embodiment, the outer contour of the housing 1 is arranged in a tubular shape. The tubular housing 1 includes an atomizing tube segment 14, a filtering tube segment 15, a top cover 16, and a bottom cover 17. One end of the filtering tube segment 15 is detachably connected to the atomizing tube segment 14. The top cover 16 covers one end of the atomizing tube segment 14 that is away from the filtering tube segment 15, and the bottom cover 17 covers one end of the filtering tube segment 15 that is away from the atomizing tube segment 14. The atomizer 5 and the vial 4 are placed in the atomizing tube segment 14, the fan 2 and the filter 3 are placed in the filtering tube segment 15, and the air outlet 12 and the outlet orifice 13 are arranged on the top cover 16, and the air inlet 11 is arranged on the filtering tube segment 15, so as to make the structural layout of the air purification-aromatherapy device compact and reasonable, which is conducive to reducing the volume of the air purification-aromatherapy device and occupying less space, thereby facilitating the placement in the narrow space of the vehicle.

According to an embodiment, referring to FIGS. 1-3, as a specific implementation of the air purification-aromatherapy device provided in the present application, an outer diameter of the atomizing tube segment 14 is greater than an outer diameter of the filtering tube segment 15.

In this embodiment, the outer diameter of the atomizing tube segment 14 is configured to be larger than the outer diameter of the filtering tube segment 15, such that the structural layout of the air purification-aromatherapy device is compact and reasonable, which is conducive to reducing the volume of the air purification-aromatherapy device and occupying less space, thereby facilitating the placement in the narrow space of the vehicle.

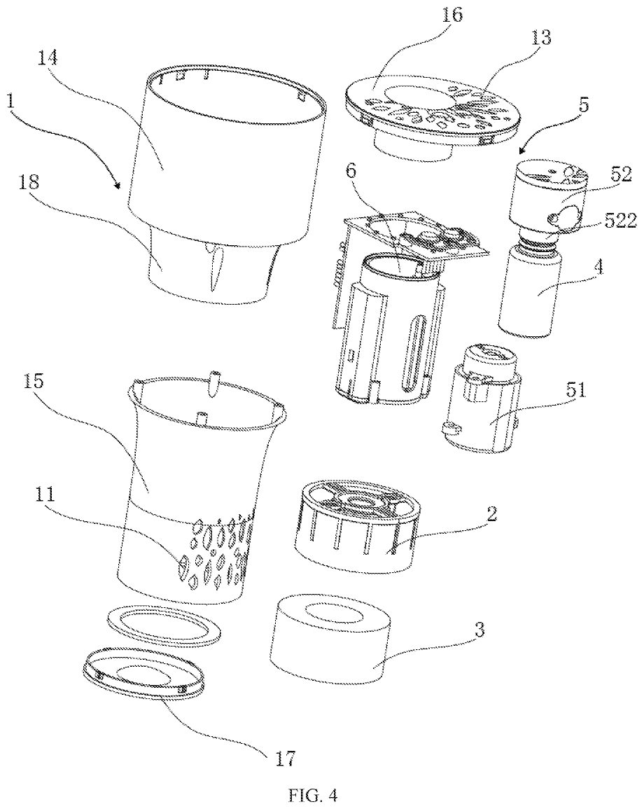

According to an embodiment, referring to FIG. 4, as a specific implementation of the air purification-aromatherapy device provided in the present application; the housing 1 further includes: a positioning tube segment 18 for cooperatively connecting the filtering tube segment 15, one end of the positioning tube segment 18 is connected to the atomizing tube segment 14, and one end of the filtering tube segment 15 is sleeved on the positioning tube segment 18.

In this embodiment, the positioning tube segment 18 is disposed at one end of the atomizing tube segment 14, when connecting the atomizing tube segment 14 and the filtering tube segment 15, only one end of the filtering tube segment 15 needs to be sleeved on the positioning tube segment 18, that is, the filtering tube segment 15 is connected to one end of the atomizing tube segment 14 by the cooperation of the positioning tube segment 18, so as to conveniently and quickly position and fix the filtering tube segment 15 to one end of the atomizing tube segment 14, thereby improving the efficiency of connection and installation.

According to an embodiment, referring to FIGS. 2-4, as a specific implementation of the air purification-aromatherapy device provided in the present application, the atomizer 5 includes an air pump 51 for providing an airflow, an atomizing hood 52 provided with an atomizing chamber 521, a gas nozzle 53 placed in the atomizing chamber 521, an air tube (not shown in the figures) connecting the air pump 51 and the gas nozzle 53, an oil suction tube 55 extending into the vial 4, and an oil nozzle 54 connected to the oil suction tube 55. The atomizing hood 52 is provided with a connecting port 522 for cooperatively connecting the vial 4. The connecting port 522 is in communication with the atomizing chamber 521. An air outlet of the gas nozzle 53 is located at a corresponding position of an oil outlet of the oil nozzle 54. And the atomizing chamber 521 is in communication with the outlet orifice 13.

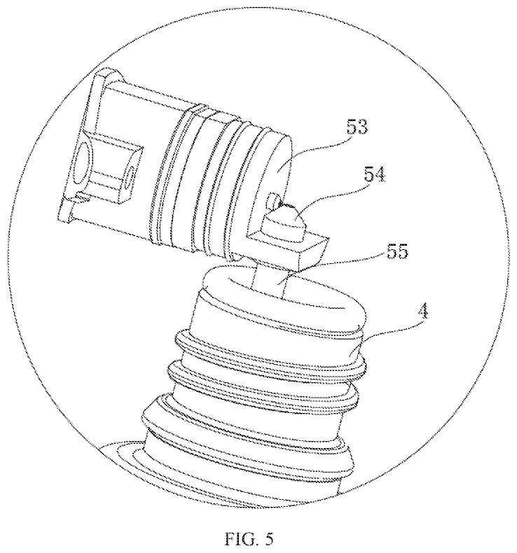

In this embodiment, the atomizing hood 52 is provided with an atomizing chamber 521 therein. The atomizing hood 52 is provided with an outlet orifice 13 thereon communicating with the atomizing chamber 521. A lower end of the atomizing hood 52 is provided with a connecting port 522. The vial mouth the vial 4 is connected to the atomizing hood 52 through the connecting port 522. The atomizing hood 52 is installed at the housing 1, the air pump 51 is installed in the housing, and the air pump 51 is supported and protected by the housing 1. One end of the air tube is connected to the air pump 51, and the other end of the air tube is connected to the gas nozzle 53. The airflow provided by the air pump 51 is transmitted to the gas nozzle 53 through the air tube and sprayed from the gas nozzle 53. An oil outlet of the oil nozzle 54 is located in the atomizing chamber 521. As shown in FIG. 5, an air outlet of the gas nozzle 53 is located at a corresponding position of the oil outlet of the oil nozzle 54, and the oil nozzle 54 is located at a corresponding position of the connecting port 522, so that when the connecting port 522 is connected to the vial 4, the oil nozzle 54 can extract essential oil from the vial 4 through the oil suction tube 55. An upper end of the oil nozzle 54 extends into the atomizing chamber 521, and the air outlet of the gas nozzle 53 is located at the upper end of the oil nozzle 54. When high-pressure gas is generated by the air pump 51 and sprayed from the gas nozzle 53, a negative pressure will be formed at the upper end of the oil nozzle 54 to extract essential oil from the vial 4 through the oil suction tube 55, and the extracted essential oil is sprayed from the oil outlet of the oil nozzle 54. The sprayed liquid droplets will be dispersed by the high-speed airflow sprayed from the gas nozzle 53 and atomized to form a mixed airflow, and at the same time, the pressure in the atomizing chamber 521 will be increased. As the connecting port 522 is connected with the vial 4, the airflow in the atomizing chamber 521 is sprayed from the outlet orifice 13, so as to realize the aroma of the air in the vehicle and achieve the purpose of improving the air quality in the vehicle.

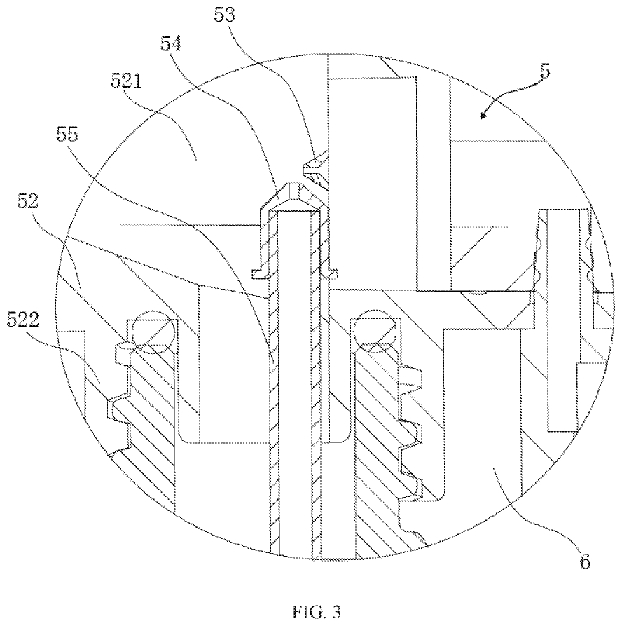

According to an embodiment, referring to FIGS. 2 and 3, as a specific implementation of the air purification-aromatherapy device provided in the present application, an outer sidewall of the vial mouth of the vial 4 is provided with external threads, and an inner sidewall of the connecting port 522 is provided with internal threads that match with the external threads.

In this embodiment, the external threads is disposed on the outer sidewall of the vial mouth of the vial 4, and the internal threads is disposed on the inner sidewall of the connecting port 522. By matching the external threads with the internal threads, the vial 4 can be connected to the connecting port 522 conveniently and quickly, and the sealing of the connection between the vial 4 and the connecting port 522 can be enhanced.

According to an embodiment, referring to FIGS. 2 and 3, as a specific implementation of the air purification-aromatherapy device provided in the present application, a bottom of the atomizing chamber 521 has an inclined surface extending obliquely to the connecting port 522.

In this embodiment, the bottom of the atomizing chamber 521 has an inclined surface that extends obliquely to the connecting port 522. By the guidance of the inclined surface, large particles of essential oil droplets are guided back to the vial 4, so as to avoid affecting the atomization effect of essential oil and reduce the waste of essential oil.

According to an embodiment, referring to FIGS. 2-4, as a specific implementation of the air purification-aromatherapy device provided in the present application, the housing 1 is provided with an accommodation chamber 6 therein for accommodating and positioning the vial 4, such that the vial 4 is accommodated and positioned in the accommodation chamber 6 to facilitate the installation of the vial 4 and enhance the stability of the installation of the vial 4.

According to an embodiment, referring to FIGS. 2-3, as a specific implementation of the air purification-aromatherapy device provided in the present application, a connecting sleeve is disposed at one end of the oil nozzle 54 far from the oil outlet, the oil suction tube 55 is detachably inserted in the connecting sleeve, and the oil suction tube 55 is placed in the vial 4.

In this embodiment, the connecting sleeve is disposed at one end of the oil nozzle 54 far from the oil outlet, and the oil suction tube 55 is detachably inserted in the connecting sleeve. A lower end of the oil nozzle 54 is connected with the oil suction tube 55, through which the essential oil in the vial 4 can be sucked to the oil nozzle 54 under the effect of negative pressure. In addition, the oil suction tube 55 is detachably inserted into the connecting sleeve of the oil nozzle 54, and the oil suction tube 55 of different length can be replaced according to different vials 4 to improve the adaptability.

According to an embodiment, referring to FIGS. 2 and 3, as a specific implementation of the air purification-aromatherapy device provided in the present application, the axial direction of the outlet of the gas nozzle 53 is directed to a top of an upper sidewall of the oil nozzle 54. Therefore, when the airflow is sprayed from the outlet of the gas nozzle 53, the airflow can cover the upper end of the oil nozzle 54 to better form a vacuum at the upper end of the oil nozzle 54. At the same time, the top of the upper sidewall of the oil nozzle 54 can change the flow direction of the airflow sprayed from the gas nozzle 53, so as to better atomize the essential oil droplets extracted by the oil nozzle 54.

According to an embodiment, referring to FIG. 5, as a specific implementation of the air purification-aromatherapy device provided in the present application, the axial direction of the outlet of the gas nozzle 53 is tilted upwardly to point to, or perpendicularly points to, the top of the upper sidewall of the oil nozzle 54. This structure can prevent the airflow sprayed from the gas nozzle 53 from being blown into the nozzle at the upper end of the oil nozzle 54. Further, the essential oil can be better extracted, and the extracted essential oil can be obliquely and upwardly blown, so as to atomize the essential oil better.

According to an embodiment, referring to FIG. 5, as a specific implementation of the air purification-aromatherapy device provided in the present application, the upper sidewall of the oil nozzle 54 is conical, so that the upper sidewall of the oil nozzle 54 can guide the airflow sprayed from the gas nozzle 53 upward, so as to enable the airflow to better atomize the essential oil extracted from the oil nozzle 54. In some other embodiments, the upper sidewall of the oil nozzle 54 may also have an upwardly-convex circular-arc shape.

The above are only preferred embodiments of the present application, and are not intended to limit the present application. Any modification, equivalent replacement, and improvement made within the spirit and principle of the present application shall be included in the protection scope of the present application.

What is claimed is:

1. An air purification-aromatherapy device, comprising:
a housing, provided with an air inlet and an air outlet;
a fan, disposed within the housing;
a filter, arranged at the air inlet;
a vial, detachably arranged in the housing for storing essential oil; and
an atomizer, configured for atomizing the essential oil in the vial and spraying out an atomized essential oil;
wherein the atomizer is disposed within the housing, and the housing is provided with an outlet orifice for spraying the atomized essential oil out of the housing.

2. The air purification-aromatherapy device according to claim 1, wherein the filter is cylindrical, an outer peripheral surface of the filter is affixed to an inner sidewall of the housing, and the air inlet is defined in a sidewall of the housing.

3. The air purification-aromatherapy device according to claim 2, wherein the air inlet comprises a plurality of air inlet holes.

4. The air purification-aromatherapy device according to claim 1, wherein an outer contour of the housing is tubular; the housing comprises: an atomizing tube segment, a filtering tube segment detachably connected to the atomizing tube segment at one end, a top cover covering one end of the atomizing tube segment that is far from the filtering tube segment, and a bottom cover covering one end of the filtering tube segment that is far from the atomizing tube segment, the atomizer and the vial are placed in the atomizing tube segment, the fan and the filter are placed in the filtering tube segment, the air outlet and the outlet orifice are arranged on the top cover, and the air inlet is arranged on the filtering tube segment.

5. The air purification-aromatherapy device according to claim 4, wherein an outer diameter of the atomizing tube segment is larger than an outer diameter of the filtering tube segment.

6. The air purification-aromatherapy device according to claim 5, wherein the housing further comprises a positioning tube segment configured for cooperatively connecting the filtering tube segment, one end of the positioning tube segment is connected to the atomizing tube segment, and one end of the filtering tube segment is sleeved on the positioning tube segment.

7. The air purification-aromatherapy device according to claim 1, wherein the atomizer comprises an air pump for providing an airflow, an atomizing hood provided with an atomizing chamber, a gas nozzle placed in the atomizing chamber, an air tube connecting the air pump and the gas nozzle, an oil suction tube extending into the vial, and an oil nozzle connected to the oil suction tube , the atomizing hood is provided with a connecting port for cooperatively connecting the vial, the connecting port is in communication with the atomizing chamber, an air outlet of the gas nozzle is located at a corresponding position of an oil outlet of the oil nozzle, and the atomizing chamber is in communication with the outlet orifice.

8. The air purification-aromatherapy device according to claim 7, wherein an outer sidewall of a vial mouth of the vial is provided with external threads, and an inner sidewall of the connecting port is provided with internal threads that match with the external threads.

9. The air purification-aromatherapy device according to claim 7, wherein a bottom of the atomizing chamber has an inclined surface extending obliquely to the connecting port.

10. The air purification-aromatherapy device according to claim 1, wherein the housing is provided with an accommodation chamber therein for accommodating and positioning the vial.
